(19) United States
(12) Patent Application Publication
(10) Pub. No.: US 2024/0254448 A1
(43) Pub. Date: Aug. 1, 2024

(54) DUAL-LAYER CULTURE SUBSTRATE

(71) Applicant: WASEDA UNIVERSITY, Tokyo (JP)

(72) Inventors: Naoya TAKEDA, Tokyo (JP); Yuji TSUCHIDO, Tokyo (JP); Mai ONUKI, Tokyo (JP)

(73) Assignee: WASEDA UNIVERSITY, Tokyo (JP)

(21) Appl. No.: 18/565,425

(22) PCT Filed: May 27, 2022
(86) PCT No.: PCT/JP2022/021748

§ 371 Date: Nov. 29, 2023

(30) Foreign Application Priority Data
Jun. 1, 2021 (JP) 2021-092013

(51) Int. Cl. C12N 5/071; C12M 1/12; C12M 3/00

(52) U.S. Cl. CPC C12N 5/0679; C12M 21/08; C12M 25/02; C12N 2503/04; C12N 2513/00; C12N 2533/54; C12N 2533/78

(57) ABSTRACT

The purpose of the present invention is to provide: a cell culture substrate, which is suitable for the fabrication of an in vitro biological tissue model of a biological tissue having an air-liquid interface and which enables temporal and real-time observation under an optical microscope, and a method for manufacturing the same; and a biological tissue model fabricated by using the cell culture substrate and a method for fabricating the same. A cellulose derivative membrane that is light-permeable under a wet condition by an aqueous medium is made porous to thereby impart water absorption properties suitable for a culture substrate thereto. This porous cellulose derivative membrane is combined with polymer microfibers to give a transparent dual-layer substrate as a cell culture substrate. Further, cells are cultured at an air-liquid interface using the transparent dual-layer substrate to give an in vitro biological tissue model having characteristics similar to a biological tissue.

### TECHNICAL FIELD

The present invention relates to a dual-layer culture substrate for cell culture and a biological tissue model fabricated by using the dual-layer culture substrate. The present invention is particularly related to a dual-layer culture substrate comprising a porous cellulose derivative membrane and polymer microfibers, which enables real-time observation and cell culture at an air-liquid interface, and to a biological tissue model fabricated by using the dual-layer culture substrate.

### BACKGROUND ART

The intestinal epithelial tissue model is useful for absorption and metabolism studies in the development of functional foods and/or drugs, and has large expectations for a tool as a substitute for the animal experiment. Conventional examples of preparing an intestinal tissue model include a method accomplished by attaching cells on a commercially-available porous membrane that is immersed in a growth medium (non-patent document 1). However, there has been an issue with the culture substrate that it is expensive and has a small culture surface, and an issue that the intestinal tissue model has insufficient functionality and structure compared to biological tissues (such as two-dimensional planer structure without any villous protrusions, low mucus production, a requirement of long period for differentiation or maturation).

The inventors developed a cost-effective dual-layer culture substrate of a commercially available paper (lower layer) on which microfibers made up of gelatin are spun by electrospinning (upper layer) (non-patent document 2). The inventors also developed a system for performing air-liquid interface culture while exposing cells to the air phase, where a culture medium is retained in a paper layer and supplied through the mesh of the microfibers from the cell basal side attached to the upper fiber layer (non-patent document 3). This dual-layer culture substrate can be used to culture intestinal epithelial cells at an air-liquid interface in order to make an intestinal epithelial tissue model having the advantages of:
(i) Forming a three-dimensional structure of intestinal villus that is analogous to the living body;
(ii) Excellency in mucus production capacity and having barrier, digestion and drug metabolism functionalities; and
(iii) Differentiation or maturation in a short period of time (for 10 to 12 days).

However, the above-mentioned culture substrate that is made in combination of paper and gelatin is incapable of performing real-time observation with an optical microscope because of the low light permeability of paper, which therefore necessitates an observation of, for example, an electron microscope after fixation of a cell or tissue sample; that is, the culture substrate has an issue that it is difficult to make a stable tissue model.

### PRIOR ART DOCUMENTS

### Non-Patent Documents

[Non-patent document 1] Thermo Fisher Scientific Inc., application note on cell culture inserts: “https://www.thermofisher.com/jp/ja/home/life-science/cell-culture/cell-culture-plastics/cell-culture-inserts.html”
[Non-patent document 2] Ozaki A, et al., Biofabrication 8 (2016) 035010, doi: 10.1088/1758-5090/8/3/035010
[Non-patent document 3] Naoya Takeda, the 18th Congress of the Japanese Society for Regenerative Medicine, abstract of program O-24-4

### SUMMARY OF THE INVENTION

### Problems to be Solved by the Invention

It is an object of the present invention to provide a dual-layer culture substrate capable of fabricating an intestinal epithelial tissue model which cultures intestinal epithelial cells to thereby enable temporal observation of culture cells using an optical microscope, and forms a conformational structure having a shape of intestinal villus that is analogous to the biological tissue, and has functionalities such as mucus productivity and various enzymic activities and a manufacturing method thereof, and to provide an intestinal epithelial tissue model fabricated by the dual-layer culture substrate and a method of fabricating the same.

### Means to Solve the Problems

According to the present invention, a porosity is imparted to the cellulose membrane under a prescribed condition to thereby provide a novel dual-layer culture substrate that is made in combination with a porous cellulose derivative membrane and polymer microfibers. The dual-layer culture substrate turns transparent when it is soaked in an aqueous medium such as a culture medium, which enables real-time and temporal observation of culture cells using an optical microscope without damaging a material to be observed such as culture cell or tissue.

Specifically, the present invention provides a transparent dual-layer substrate for culturing a cell and/or a tissue, comprising a porous cellulose derivative membrane on which polymer microfibers are spun and laminated, wherein the porous cellulose derivative membrane is light-permeable under a wet condition

In the transparent dual-layer substrate according to the present invention, the culturing may be an air-liquid interface culture.

In the transparent dual-layer substrate according to the present invention, the cell may be an intestinal epithelial cell or an epithelial cell of another tissue or organ or the cell may be an epidermis cell, and wherein the tissue may be an intestinal epithelial tissue or another epithelial tissue or the tissue may be an epidermis tissue.

In the transparent dual-layer substrate according to the present invention, the porous cellulose derivative membrane may be of a material selected from cellulose acetate, cellulose nitrate and a regenerated cellulose.

In the transparent dual-layer substrate according to the present invention, the polymer microfibers may be of a material selected from gelatin, polysaccharide, collagen, polycaprolactone, polylactic acid, polyglycolic acid, poly-p-dioxanone, polyhydroxybutyric acid, trimethylene carbonate, polyacrylic or polymethacrylic acid derivative or a copolymer thereof; or water-soluble polymers of polyvinyl alcohol, polyethylene oxide, polyacrylamide, polyvinylpyrrolidone, dextran or polyethylene glycol, or a cross-linked material thereof; or a mixture thereof.

In the transparent dual-layer substrate according to the present invention, the polysaccharide may be cellulose, an oxidized derivative of cellulose, chitin, chitosan, agarose or carrageenan.

The present invention also provides a method of manufacturing a transparent dual-layer substrate for culturing a cell and/or a tissue, comprising the steps of:
1) dissolving a cellulose derivative in an organic solvent to prepare an organic solvent solution of the cellulose derivative;
2) coating a substrate with the organic solvent solution of the cellulose derivative and drying it to prepare a cellulose derivative membrane;
3) drying the cellulose derivative membrane for a prescribed period of time, and then immersing it in hot water for a prescribed period of time, and then immersing it in cold water for a prescribed period of time, and then peeling it from the substrate to prepare a porous cellulose derivative membrane; and
4) spinning polymer microfibers on the porous cellulose derivative membrane by electrospinning to laminate the membrane with the polymer microfibers.

In the manufacturing method according to the present invention, the period of time for the drying may be 10 seconds under a temperature of 20 to 25° C., the period of time for the immersing in hot water may be 10 minutes under a temperature of 80° C., and the period of time for the immersing in cold water may be 60 minutes under a temperature of 20 to 25° C.

In the manufacturing method according to the present invention, the cell may be an intestinal epithelial cell or an epithelial cell of another tissue or organ or the cell may be an epidermis cell, and the tissue may be an intestinal epithelial tissue or another epithelial tissue or the tissue may be an epidermis tissue.

In the manufacturing method according to the present invention, the porous cellulose derivative membrane may be selected from cellulose acetate, cellulose nitrate or a regenerated cellulose.

In the manufacturing method according to the present invention, the polymer microfibers may be of a material selected from gelatin, polysaccharide, collagen, polycaprolactone, polylactic acid, polyglycolic acid, poly-p-dioxanone, polyhydroxybutyric acid, trimethylene carbonate, polyacrylic or polymethacrylic acid derivative or a copolymer thereof; or water-soluble polymers of polyvinyl alcohol, polyethylene oxide, polyacrylamide, polyvinylpyrrolidone, dextran, or polyethylene glycol, or a cross-linked material thereof; or a mixture thereof.

In the manufacturing method according to the present invention, the polysaccharide may be cellulose, an oxidized derivative of cellulose, chitin, chitosan, agarose or carrageenan.

In the manufacturing method according to the present invention, the substrate may be a glass substrate.

The present invention also provides a biological tissue model fabricated by culturing a cell and/or a tissue on polymer microfibers using the transparent dual-layer substrate.

In the biological tissue model according to the present invention, the culturing may be an air-liquid interface culture.

In the biological tissue model according to the present invention, the cell may be an intestinal epithelial cell or an epidermis cell, the tissue may be an intestinal epithelial tissue or an epidermis tissue, and the biological tissue model may be selected from an intestinal epithelial tissue model and an epidermis tissue model.

In the biological tissue model according to the present invention, the intestinal epithelial tissue model may express intestinal villus structure, microvillus structure, digestive enzyme activity, drug-metabolizing enzyme activity, mucus production capacity, barrier function and/or alkaline phosphatase (ALP).

The present invention also provides a method of fabricating a biological tissue model, wherein the method comprises culturing a cell and/or a tissue on polymer microfibers using the transparent dual-layer substrate.

In the method of fabricating a biological tissue model according to the present invention, the culturing may be an air-liquid interface culture.

In the method of fabricating a biological tissue model according to the present invention, the cell may be an intestinal epithelial cell or an epidermis cell, the tissue may be an intestinal epithelial tissue or an epidermis, tissue, and the biological tissue model may be selected from an intestinal epithelial tissue model and an epidermis tissue model.

In the method of fabricating a biological tissue model according to the present invention, the intestinal epithelial tissue model may have intestinal villus structure, microvillus structure, digestive enzyme activity, drug-metabolizing enzyme activity and/or mucus production capacity.

### Effects of the Invention

The culture system using the transparent dual-layer substrate enables the fabrication of a substrate having a large area. This dual-layer culture substrate has excellent workability where a large substrate can be cut into a suitable size for use. The system can be also expected to be applied to the culturing of various epithelial cells and to the formation of biological tissues. These biological tissue models can be developed into tissue models for drug development or for evaluating the absorption or metabolism of functional foods or as a tissue for implant therapy.

### BRIEF DESCRIPTION OF THE DRAWINGS

FIG. 1 shows a picture illustrating a cross-sectional structure of a dual-layer substrate of a cellulose acetate film (hereafter referred to as “CA film P”) that is observed by a scanning electron microscope and prepared with the condition of: “10 seconds of drying”, “immersing it in hot water at 80° ° C. for 10 minutes”, “immersing it in cold water at 20 to 25° C. for 60 minutes” and a “thickness of 200 μm”.

FIG. 2 shows images of the CA film P and fibers which were observed by a scanning electron microscope.

FIG. 3 shows images illustrating the results of stability examination on fiber diameters of the CA film P substrate in aqueous media.

FIG. 4 shows images of samples using transparent dual-layer substrates on which Caco-2 had been subjected to submerged static culture until 21 days later (up to Day 21) after the initiation of culturing, which were then observed using a phase contrast microscope.

FIG. 5 illustrates pictures showing fluorescence microscope observation images of cultured samples in which Caco-2 were subjected to a submerged static culture which had been observed for 21 days (up to Day 21). Hoechst 33342 was used to stain nuclei, and Alexa Fluor 568 phalloidin was used to stain actin.

FIG. 6 illustrates pictures showing phase contrast images of cultured cells and/or tissue samples having been temporally observed until 21 days later (Day 21) after Caco-2 cells were cultured by air-liquid interface culture using the transparent dual-layer substrate of the present invention.

FIG. 7 illustrates pictures showing phase contrast images of three-dimensional structures at the days of 10 days later (Day 10), 12 days later (Day 12) and 21 days later (Day 21) after Caco-2 cells were cultured by air-liquid interface culture on the dual-layer substrate of the present invention, where the amount of solution of the gelatin fiber to be spun on the dual-layer substrate of the present invention had been varied from 1.5 mL to 3.0 mL. The scale bars indicate 100 μm.

FIG. 8 illustrates pictures that represent the images of nuclei and actin and a superimposed merged image of them, having been observed by a confocal microscope, which show the morphology of Caco-2 cells on the day 21 days later after the initiation of the culture, where the amount of solution of the gelatin fiber to be spun on the dual-layer substrate of the present invention had been varied from 1.5 mL to 3.0 mL.

FIG. 9 illustrates pictures showing morphological observation images of Caco-2 cells that had been cultured up to the day 21 days later after the Caco-2 cells were cultured by air-liquid interface culture on the dual-layer substrate in which an amount of 2.5 mL solution of gelatin fiber was spun on the CA film P.

FIG. 10A shows SEM images of the samples each obtained with an air-liquid interface culture on a dual-layer substrate of CA film Player and gelatin fibers.

FIG. 10B shows SEM images of the samples each obtained with a submerged culture on a dual-layer substrates of CA film Player and gelatin fibers.

FIG. 10C shows SEM images of the samples each obtained by an air-liquid interface culture using a cell culture insert that is a commercially-available culture substrate.

FIG. 10D shows SEM images of the samples each obtained by a submerged culture using the cell culture insert.

FIG. 11 shows the progresses of ANPEP activity when Caco-2 cells were subjected to an air-liquid interface culture or a submerged culture using the dual-layer substrate of the present invention or the cell culture insert. In the figure, “Dual-layer substrate/Air-liquid” indicates temporal progress in ANSEP activity when an air-liquid interface culture using the dual-layer substrate of the present invention had been carried out, “Dual-layer substrate/Submerged” indicates a temporal progress in ANSEP activity when a submerged culture using the dual-layer substrate of the present invention had been carried out, “Insert/Air-liquid” indicates a temporal progress in ANSEP activity when an air-liquid interface culture using the culture insert had been carried out, and “Insert/Submerged” indicates a temporal progress in ANSEP activity when a submerged culture using the culture insert had been carried out.

FIG. 12 illustrates the progresses of CYP3A4 activity when Caco-2 cells were subjected to an air-liquid interface culture or a submerged culture using the dual-layer substrate of the present invention or a cell culture insert. In the figure, “Dual-layer substrate/Air-liquid” indicates a case in which an air-liquid interface culture using the dual-layer substrate of the present invention had been carried out, “Dual-layer substrate/Submerged” indicates a case in which a submerged culture using the dual-layer substrate of the present invention had been carried out, “Insert/Air-liquid” indicates a case in which an air-liquid interface culture using the culture insert had been carried out, and “Insert/Submerged” indicates a case in which a submerged culture using the culture insert had been carried out.

FIG. 13A illustrates pictures showing observed images by an optical microscope of Caco-2 cells that were subjected to an air-liquid interface culture or a submerged culture using the dual-layer substrate of the present invention or a cell culture insert, and then stained by alcian blue which serves to stain mucus into blue. In the figure, “Dual-layer substrate/Air-liquid” indicates an optical microscope image when an air-liquid interface culture using the dual-layer substrate of the present invention had been carried out, “Dual-layer substrate/Submerged” indicates an optical microscope image where a submerged culture using the dual-layer substrate of the present invention had been carried out, “Insert/Air-liquid” indicates an optical microscope image where an air-liquid interface culture using the culture insert had been carried out, and “Insert/Submerged” indicates an optical microscope image where a submerged culture using the culture insert had been carried out.

FIG. 13B illustrates the progresses of mucus production capacity which had been temporally measured when Caco-2 cells were subjected to an air-liquid interface culture or a submerged culture using the dual-layer substrate of the present invention or a cell culture insert. In the figure, “Dual-layer substrate/Air-liquid” indicates a temporal progress in mucus productivity when an air-liquid interface culture using the dual-layer substrate of the present invention had been carried out, “Dual-layer substrate/Submerged” indicates a temporal progress in mucus productivity when a submerged culture using the dual-layer substrate of the present invention had been carried out, “Insert/Air-liquid” indicates a temporal progress in mucus productivity when an air-liquid interface culture using the culture insert had been carried out, and “Insert/Submerged” indicates a temporal progress in mucus productivity when a submerged culture using the culture insert had been carried out.

FIG. 14A shows pictures of chambers for measuring TEER values that represent barrier function.

FIG. 14B illustrates the measured results of TEER values. In the figure, “CA dual-layer substrate/Air-liquid” indicates a temporal progress in TEER value when an air-liquid interface culture using the dual-layer substrate of the present invention had been carried out, “CA dual-layer substrate/Submerged” indicates a temporal progress in TEER value when a submerged culture using the dual-layer substrate of the present invention had been carried out, “Insert/Air-liquid” indicates a temporal progress in TEER value when an air-liquid interface culture using the culture insert had been carried out, and “Insert/Submerged” indicates temporal progress in TEER value when a submerged culture using the culture insert had been carried out.

### MODE FOR CARRYING OUT THE INVENTION

### 1. Transparent Dual-Layer Substrate

One embodiment of the present invention is a transparent dual-layer substrate for culturing a cell and/or a tissue, comprising a porous cellulose derivative membrane that is light-permeable under a wet condition, wherein polymer microfibers are spun to be laminated on the porous cellulose derivative membrane.

In the transparent dual-layer substrate according to the present invention, the culturing may be preferably an air-liquid interface culture but may not particularly be limited to the air-liquid interface culture. The culturing may alternatively be a submerged culture.

The transparent dual-layer substrate according to the present invention is a dual-layer culture substrate comprising of a porous cellulose derivative membrane (lower layer) and polymer microfibers (upper layer). The porous cellulose derivative membrane can retain a culture medium to supply the medium through the mesh of the microfibers to the basal side of the cells that are attached to the upper fiber layer, which therefore enables not only a submerged culture but also an air-liquid interface culture while being exposed to air. As shown in the following working examples, the characteristics of a cultured cell and/or tissue differ depending on a choice between the submerged culture and the air-liquid interface culture. For example, the activity of the alanine aminopeptidase (ANPEP)—a digestive enzyme—is higher when they are cultured by air-liquid interface culture compared to the one being cultured by a submerged culture.

Further, the dual-layer culture substrate turns into a transparent and transparent dual-layer substrate when it is in contact with an aqueous medium such as a culture medium, which thereby enables the cultured cells to be observed by an optical microscope.

The transparent dual-layer substrate according to the present invention may readily be prepared in a conventional laboratory facility with simple equipment if there are an electrospinning device and a film orientation device. The transparent dual-layer substrate according to the present invention is also cost effective, and theoretically enables the fabrication of a substrate having a large area. The substrate has superiority in these respects over commercially available culture inserts.

In the transparent dual-layer substrate, although not limited to the followings, the cell may be an intestinal epithelial cell or an epidermis cell, and the tissue may be an intestinal epithelial tissue or an epidermis tissue.

The dual-layer substrate may be used to culture an intestinal epithelial cell or an epidermis cell in order to produce a biological model such as an intestinal epithelial tissue model or an epidermis tissue model. The transparent dual-layer substrate may allow continual microscope observation in real-time during the culturing. Further, the dual-layer culture substrate comprising a cellulose derivative membrane and polymer microfibers may be used to more efficiently and stably enable tissue formation of a biological model at an enhanced yield compared to a conventionally used dual-layer substrate made up of the combination of paper and gelatin.

In the transparent dual-layer substrate, a material of the porous cellulose derivative membrane may be selected from cellulose acetate, cellulose nitrate and a regenerated cellulose, and the material is preferably cellulose acetate.

In the transparent dual-layer substrate, the polymer microfibers may be of a material selected from gelatin, polysaccharide, collagen, polycaprolactone, polylactic acid, polyglycolic acid, poly-p-dioxanone, polyhydroxybutyric acid, trimethylene carbonate, polyacrylic or polymethacrylic acid derivative or a copolymer thereof; or water-soluble polymers of polyvinyl alcohol, polyethylene oxide, polyacrylamide, polyvinylpyrrolidone, dextran or polyethylene glycol, or a cross-linked material thereof; or a mixture thereof. The polymer microfibers may preferably be of gelatin.

In the transparent dual-layer substrate, as an example of the polysaccharide, cellulose, an oxidized derivative of cellulose, chitin, chitosan, agarose or carrageenan is preferred.

### 2. Manufacturing Method of Transparent Dual-Layer Substrate

Another embodiment of the present invention is directed to a method of manufacturing a transparent dual-layer substrate. The manufacturing method includes the steps of manufacturing a porous cellulose derivative membrane, and spinning polymer microfibers on the porous cellulose derivative membrane by electrospinning to laminate it with polymer microfibers.

Specifically, the method of manufacturing the transparent dual-layer substrate is a method of manufacturing a transparent dual-layer substrate for culturing a cell and/or a tissue, comprising the steps:
1) dissolving a cellulose derivative in an organic solvent to prepare an organic solvent solution of the cellulose derivative;
2) coating a substrate with the organic solvent solution of the cellulose derivative and drying it to prepare a cellulose derivative membrane;
3) drying the cellulose derivative membrane for a prescribed period of time, and then immersing it in hot water for a prescribed period of time, and then immersing it in cold water for a prescribed period of time, and then peeling the membrane from the substrate to prepare a porous cellulose derivative membrane; and
4) spinning polymer microfibers on the porous cellulose derivative membrane by electrospinning.

Examples of the method for performing the coating include preferably a bar coating method and a casting method which are not particularly limited. The coating film has a thickness of 150 to 200 μm, which is not particularly limited.

In the manufacturing method, the period of time for performing the drying may be 10 to 300 seconds, preferably 5 to 15 seconds, more preferably 10 seconds, and the temperature for performing the drying is room temperature which is specifically 15 to 30° C., and preferably 20 to 25° C. Moreover, the temperature for immersing it in hot water is 7 to 100° C., preferably 70 to 80° C., and more preferably 80° C. The period of time for immersing it in hot water is 5 to 60 minutes, preferably 10 to 30 minutes, and more preferably 10 minutes. Further, the temperature for immersing it in cold water is preferably 20 to 25° C.

The period of time for immersing it in cold water is preferably 30 to 90 minutes, and more preferably 60 minutes.

That is, in the manufacturing method, it is preferred that the period of time for performing the drying be 10 seconds under a temperature of 20 to 25° C., that the period of time for the immersing in hot water be 10 minutes under a temperature of 80° C., and that the period of time for the immersing in cold water be 60 minutes under a temperature of 20 to 25° C.

In the manufacturing method, although not limited to the following, it is preferred that the cell be an intestinal epithelial cell or an epithelial cell of another tissue or organ or the cell be of an epidermis cell, and that the tissue be an intestinal epithelial tissue or another epithelial tissue or the tissue be an epidermis tissue although they are not limited to these.

In the manufacturing method, the porous cellulose derivative membrane is selected from cellulose acetate, cellulose nitrate and a regenerated cellulose although they are not limited to these.

In the manufacturing method, the polymer microfibers may be of a material selected from gelatin, polysaccharide, collagen, polycaprolactone, polylactic acid, polyglycolic acid, poly-p-dioxanone, polyhydroxybutyric acid, trimethylene carbonate, polyacrylic or polymethacrylic acid derivative or a copolymer thereof, or water-soluble polymers of polyvinyl alcohol, polyethylene oxide, polyacrylamide, polyvinylpyrrolidone, dextran or polyethylene glycol, or a cross-linked material thereof; or a mixture thereof although it is not limited to these.

In the manufacturing method, as an example of the polysaccharide, cellulose, an oxidized derivative of cellulose, chitin, chitosan, agarose or carrageenan is preferred.

In the manufacturing method, the substrate is preferably a glass substrate but the substrate is not particularly limited to it.

### 3. Biological Tissue Model

Another embodiment of the present invention is directed to a biological tissue model. The biological tissue model is fabricated by culturing a cell and/or a tissue on the polymer microfibers using the transparent dual-layer substrate.

In the biological tissue model, the culturing is preferably an air-liquid interface culture but the culturing is not particularly limited to it.

In the biological tissue model, it is preferred that the cell is preferably an intestinal epithelial cell or an epithelial cell of another tissue or organ or the cell be of an epidermis cell; the tissue is preferably an intestinal epithelial tissue or another epithelial tissue or the tissue be an epidermis tissue; and the biological tissue model is preferably selected from an intestinal epithelial tissue model, a tissue model of another epithelial tissue model and an epidermis tissue model.

It is preferred that the intestinal epithelial cell be a Caco-2 cell but the cell is not particularly limited to it.

In the biological tissue model according to the present invention, the intestinal epithelial tissue model has characteristics that are analogous to the characteristics of the living intestinal epithelial tissue, and is a biological tissue model specifically having intestinal villus structure, microvillus structure, digestive enzyme activity, drug-metabolizing enzyme activity and/or mucus production capacity Accordingly, the biological tissue model according to the present invention can be used not only to study drug metabolism but also to study absorption/metabolism in place of in-vivo animal experiments in the development of functional foods and/or drugs. A tissue of the biological tissue model may also be used for the application to a living body.

### 4. Method of Fabricating Biological Tissue Model

Another embodiment of the present invention is directed to a method of fabricating a biological tissue model. The method is a method of fabricating a biological tissue model which uses the transparent dual-layer substrate to culture a cell and/or a tissue on the polymer microfibers.

In the method of fabricating a biological tissue model, the culturing is preferably an air-liquid interface culture but the culturing is not particularly limited to it.

In the method of fabricating a biological tissue model, it is preferred that the cell be an intestinal epithelial cell or an epithelial cell of another tissue or organ or the cell be of an epidermis cell, that the tissue be an intestinal epithelial tissue or another epithelial tissue or the tissue be an epidermis tissue, and that the biological tissue model be selected from an intestinal epithelial tissue model, a tissue model of another epithelial tissue and an epidermis tissue model.

In the method of fabricating a biological tissue model, the intestinal epithelial tissue model has intestinal villus structure, microvillus structure, digestive enzyme activity, drug-metabolizing enzyme activity and/or mucus production capacity. Accordingly, the method of fabricating a biological tissue model according to the present invention allows one to fabricate an intestinal epithelial tissue model having characteristics that assimilate to an intestinal epithelial tissue of a living body. Further, the tissue as fabricated in accordance with the present fabrication method may be used for an application to the tissue implantation into a living body.

Examples will be described in detail below. All documents mentioned in this specification are incorporated by reference in their entirety. The examples described herein are illustrative of embodiments of the invention and should not be construed as limiting the scope of the invention.

### WORKING EXAMPLES

### (Example 1) Fabrication of Porous and Highly Transparent Cellulose Acetate Film and Manufacturing of Dual-Layer Substrate Comprising of the Film and Gelatin Fibers

A porous and highly transparent cellulose acetate film was prepared, and a dual-layer substrate serving as a culture scaffold of intestinal epithelial cells in which gelatin fibers were spun on the prepared cellulose acetate film was fabricated.

A porous and highly transparent cellulose acetate film capable of holding a culture medium was used in a liquid phase and gelatin fibers serving as a cell attachment scaffold were used as in an air phase. Attempts were made to fabricate the dual-layer substrate by spinning the gelatin fibers using electrospinning on the porous cellulose acetate film. Further, attempts were also made to strengthen the crosslinking of the gelatin fibers with themselves and with the porous cellulose acetate film since the cell culture examination takes a long period of time.

### 1-1 Reagents

1-1-1. The Following Reagents were Used for the Preparation of the Porous Cellulose Acetate Film.

(1) Cellulose acetate: FUJIFILM Wako Pure Chemical Corporation, OSAKA.

(2) Acetone: FUJIFILM Wako Pure Chemical Corporation, OSAKA.

(3) Formamide: FUJIFILM Wako Pure Chemical Corporation, OSAKA.

1-1-2. The Following Reagents were Used for the Preparation of the Dual-Layer Substrate

(1) Nitta Gelatin, beMatrix* Gelatin LS-H (Alkaline-treated gelatin derived from porcine skin), Nitta Gelatin Inc., Osaka.

(2) 1,1,1,3,3,3-Hexafluoropropan-2-ol(HFIP): Cat #PC4750, Apollo Scientific, UK.

(3) 25% glutaraldehyde solution: Cat #073-00536, FUJIFILM Wako Pure Chemical Corporation, OSAKA

### 1-2. Devices

1-2-1. The Following Devices were Used for the Preparation of the Porous Cellulose Acetate Film.

(1) Glass plate (15 cm×20 cm×5 mm): SANSYO Co., LTD.

(2) Film applicator with film thickness control functionality 150 mm: Allgood

(3) Constant-temperature bath, Cat #FTB-01: Tokyo Garasu Kikai Co., Ltd.

(4) Heat drying moisture analyzer: Cat #MOC63u, Shimadzu Corporation.

2.2 the Following Devices were Used for the Preparation of the Dual-Layer Substrate

(1) Electrospinning device: Cat #NANON-03, MECC CO., LTD., FUKUOKA.

(2) Tubeless Spinneret_75: Cat #S-TU/75, MECC CO., LTD., FUKUOKA.

(3) Drum Collector_q200W200 (Diameter 200 mm, Width 200 mm): Cat #C-DR/D200W200, MECC CO., LTD., FUKUOKA.

(4) Terumo Syringe™ 1 mL for Tuberculin, Electron beam sterilized: Cat #SS-01T, Terumo Corporation, Tokyo.

(5) Terumo Injection Needle (electron beam sterilized, 22G×1 1/4): Terumo Corporation, Tokyo.

(6) 3M® Masking Tape 243J Plus: 3M Japan Limited, Tokyo.

(7) Magnetron sputtering system: Cat #MSP-10, Vacuum device Corp., Ibaraki.

(8) Scanning electron microscope: Cat #VE-9800, KEYENCE CORPORATION, Osaka.

### 1-3. Preparation of Porous and Highly Transparent Cellulose Acetate Film

The cellulose acetate film cannot be used for a liquid-phase substrate that holds a culture medium unless the film has a good water-absorbing property. However, a method of fabricating cellulose acetate film of a prior study of the Loeb-Sourirajan method (OYA Haruhiko, “Sourirajan and the invention of asymmetric membrane”, MRC newsletter, No. 30, 2003) could not make a porous cellulose acetate film. Accordingly, attempts were made to make the cellulose acetate film porous.

### 1-3-1. Experiment

Into a screw tube bottle were prepared cellulose acetate, formamide and acetone at the weight ratio (g) of 3:5:10, respectively. The cellulose acetate solution put in the screw tube bottle was stirred for one night under room temperature. On the next day, the cellulose acetate solution was elongatedly cast onto a glass plate. The cellulose acetate solution was then immediately stretched using a film applicator with film thickness control functionality to form a film. The film was dried on the glass plate, which was then immersed in a constant-temperature bath having hot Milli-Q water. The film with glass plate was removed therefrom, and then immersed in a bath having a room-temperature Milli-Q water after which the glass plate was taken therefrom to remove the cellulose acetate film from the glass plate with tweezers, and the moisture of the cellulose acetate film was wiped out with a paper towel. The conditions for making a porous and highly transparent cellulose acetate film were studied with respect to the four items of criteria which are: thickness, drying time, hot-water treatment and cold-water treatment. A cellulose acetate film having been air-dried for one night or more was split with tweezers whilst being immersed in liquid nitrogen, and performed an Au Sputtering (Target: Au, Discharge current: 35 mA, Sputtering time: 0.5 minutes) under vacuum for the observation with a scanning electron microscope.

Moreover, in order to study the absorbency of the resultant porous cellulose acetate film, the cellulose acetate film was cut into the size of 6×6 cm, immersed into a φ100 mm dish having 20 mL of Milli-Q water, and incubated one night with a parafilm being wound around. It was then taken out on the next day to measure a water-absorbing ratio using a heat drying moisture analyzer.

Further, as used herein, a side of the resultant cellulose acetate film exposed to the air is referred to as a front side while a side of the film in contact with the glass plate is referred to as a back side.

### 1-3-2. Experimental Results

The cellulose acetate (CA) films as prepared in accordance with the respective items of criteria for the fabrication conditions (film thickness, drying time, hot-water treatment and cold-water treatment) were observed using a scanning electron microscope to study the conditions therefore.

First, three samples (Nos. 1 to 3) which respectively underwent drying for 30 seconds that is a drying period of the prior study, 60 seconds and 300 seconds were prepared. As the cold-water treatment was for solvent elimination, only a cold-water treatment was performed after the drying without performing a hot-water treatment. The thicknesses were all set as 200 μm which was the thickness of the prior study. The cellulose acetate film No. 1 having been prepared with a drying period of 30 seconds had convexities and concavities while the other two had a flat surface (not shown) with no convexities or concavities, which indicates that a shorter drying time more likely tends to result in a porous structure. In view of this, the drying time was set shorter from 30 seconds to 10 seconds to make a further consideration in the following.

Next, the period of time for the hot-water treatment will be discussed. In the treatment, the hot water had a temperature of 80° C. Three samples (Nos. 4 to 6) which respectively underwent hot-water treatments for 10 minutes, 5 minutes and 30 minutes were prepared. The drying time and the thickness were respectively set as 30 seconds and 200 μm which are the same as those of the prior study.

No significant changes were observed in the respective samples in association with a change in the time period for the hot-water treatment, and the samples did not turn into a porous structure (not shown in the figure). Accordingly, the cellulose acetate films were prepared with the hot-water treatment at 80° ° C. for 10 minutes in the following.

Next, cellulose acetate films were prepared with the drying time being set at 10 seconds. Three samples of sample No. 7 which underwent 10 seconds of drying and the cold-water treatment for only the solvent elimination, sample No. 8 which additionally underwent the hot-water treatment, and sample No. 9 whose drying time was set shorter from 10 seconds were prepared. The thicknesses were all set as 200 μm.

When the drying time was set shorter than 30 seconds which is the time of the prior study, a clear rough texture was observed on the surface, and particularly a clear porous structure was observed in sample No. 8. Sample No. 8 which additionally underwent a hot-water treatment showed a more clear porous structure than sample No. 7 which solely underwent a cold-water treatment (not shown in the figure). Accordingly, it can be inferred that the conditions of the porous structure are: “10 seconds of drying”, “treatment at 80° C. for 10 minutes” and “thickness of 200 μm”.

The cellulose acetate film sample No. 8 having been prepared in accordance with the condition of the porous structure was opaque and while, which did not pass the light and was thus unsuitable for the temporal observation. Accordingly, a test was performed by setting the thickness thereof smaller than 200 μm to see if a transparent and porous cellulose acetate film can be fabricated.

Samples Nos. 11 and 12 having thicknesses of 50 μm and 100 μm that are smaller than 200 μm were prepared. A sample No. 10 with a drying time set shorter than the case in the condition of the porous structure with the thickness of 200 μm was also prepared.

It turned out that samples Nos. 11 and 12 having a thickness smaller than 200 μm did not turn into a porous structure (not shown in the figure) even when the samples were prepared in accordance with the condition of porous structure at the thickness of 200 μm. Moreover, even when a drying time was set shorter as in the case of sample No. 10, no pore has been formed (not shown in the figure) when the thickness was smaller than 200 μm. Further, since a thinner film more likely tends to be soft like a Saran Wrap, and easily stick to a plastic, it turned out that such film is short in strength and therefore difficult to use when used as a dual-layer substrate

A cellulose acetate film No. 13 having a thickness of 150 μm with the porous condition had a transparent part and a white part, which were observed to see if there was any difference between the white and transparent parts. The cellulose acetate films having been prepared in accordance with the porous condition were visually observed. As it turned out, there were differences in the appearance of the surface between the front side and the back side of film (not shown in the figure), and therefore they were distinctively observed from each other using a scanning electron microscope.

In sample No. 13, no pore was observed in the transparent part but the white part was porous. This result indicates that the cellulose acetate film turns white and opaque when it has a porous structure while the film turns clear and colorless when it has no porous structure. In sample No. 14, the front side (a side being exposed to air) was smooth in appearance and was not porous while the back side (a side in contact with the glass plate) was matted with no gloss in appearance and was porous. Accordingly, a plurality of conditional requirements was combined to conduct examinations to see if a porous and transparent cellulose acetate film could be fabricated.

The results of the samples Nos. 7 and 9 showed that the drying time for the case of cold-water treatment of 5 seconds was more suitable than the case of 10 seconds. For this reason, samples Nos. 15 to 17 having thinner thicknesses and a drying time of 5 seconds were prepared.

As a result, a thickness smaller than 200 μm resulted in a transparent film but no pore was formed therein despite a shorter drying time. Since sample No. 13 showed a part having a porous structure despite the thickness of 150 μm, cellulose acetate films of 150 μm to 200 μm in steps of 10 μm were prepared to study if there is any better thickness that turns it into a porous and transparent film.

The cellulose acetate films of 150 μm to 200 μm in steps of 10 μm were prepared, and they had a transparent part but showed no pore when the thicknesses were smaller than 200 μm as is the same with the above results, while the thickness close to 200 μm led to a film that is white and has a pore. The current optimized fabrication conditions of the cellulose acetate films are thus “10 seconds of drying”, “treatment at 80° C. for 10 minutes” and “thickness of 200 μm” which led to a white and opaque film.

A study to see if the cold-water treatment for solvent elimination is necessary or not was made by setting the porous conditions of “10 seconds of drying”, “treatment at 80° C. for 10 minutes” and “thickness of 200 μm”. A scanning electron microscope (SEM) was used to observe and compare a cellulose acetate film as prepared with the porous conditions of “10 seconds of drying”, “treatment at 80° ° C. for 10 minutes” and “thickness of 200 μm” with a cellulose acetate film as prepared by performing a cold-water treatment at 20 to 25° C. for 60 minutes after performing the hot-water treatment (“10 seconds of drying”, “treatment at 80° C. for 10 minutes”, “treatment at 20 to 25° ° C. for 60 minutes” and “thickness of 200 μm”). The one having a cold-water treatment showed a clearer porous structure (not shown in the figure). Accordingly, it was determined to perform a cold-water treatment to eliminate a solvent of the cellulose acetate film since the culture substrate employs a dual-layer substrate having the prepared cellulose acetate film on which gelatin fibers were spun.

In the following discussion, a cellulose acetate film having been fabricated using the porous condition of “10 seconds of drying”, “treatment at 80° C. for 10 minutes”, “treatment at 20 to 25° C. for 60 minutes” and “thickness of 200 μm” will be referred to as “CA film P”

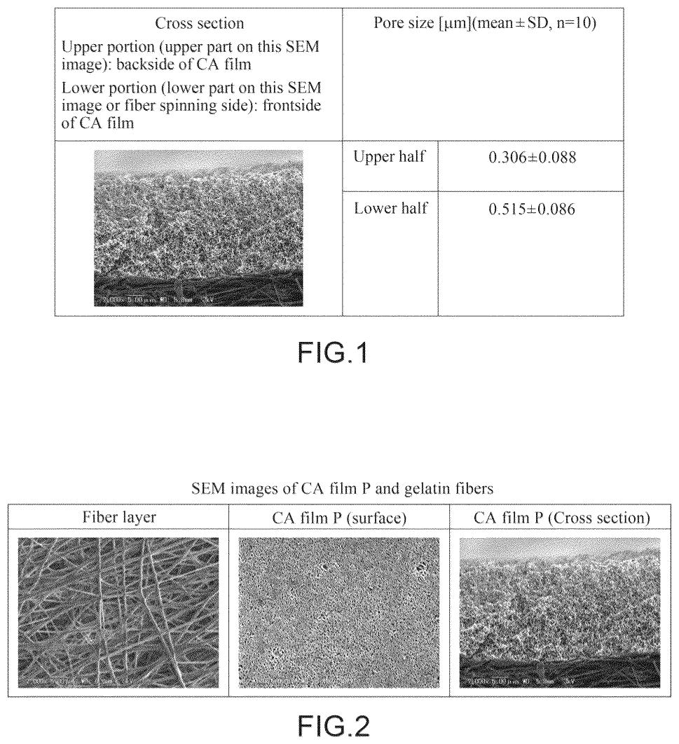

In order to assess the front-and-back sides thereof, a dual-layer substrate in which the fabricated CA film P was spun with gelatin fibers on the back side thereof was immersed in liquid nitrogen to observe a cracked section using a scanning electron microscope whose observed image is as shown in FIG. 1. It shows that the front side had a small pore size while the back side had a large pore size. It can therefore be inferred that the fibers may be spun on the front side having a smaller pore size while the back side having a pore size that is larger than the one of the front side may be set to face a liquid phase for immersing it in the culture medium in order to stably supply the culture media.

The water-absorbing ratio of the above-mentioned CA film P was measured. Table 1 shows the results. These results are the results of direct measurement of the water content without wiping out the surface moisture but simply draining the moisture at the end of the dishes because the subject intended to be measured was only moisture content contained therein and the wiping of the surface moisture involves hand working, which resulted in difference in, for example, the application of the force and therefore led to a difference in the results when the respective samples were sandwiched by paper towels of CRECIA™ to wipe out the surface moisture for subjecting them in a heat drying moisture analyzer for the measurement as did initially. The CRECIA and a porous dialysis membrane (MWCO: 6000-8000) were also measured for comparison purposes.

The measurement of the CA film P was performed such that the back side thereof was in contact with the liquid since fibers were spun on the front side of the CA film P. All of the samples had a size of 6×6 cm, and a single sheet of the sample and 20 mL of sterile water were put into a @100 mm dish, and then incubated for one night, which was then subjected to the measurement by a heat drying moisture analyzer. The qualities of water-absorbing ratios were in the order of the paper towel (CRECIA), CA film P where the back side faces underside, and the porous dialysis membrane (MWCO:6000-8000).

TABLE 1
Water absorbency examination (mean ± SD, n = 3)

| | Weight | | |
|---|---|---|---|
| | after | | |
| | removal | Weight | Water- |
| | from | after | absorbing |
| | water | heating | ratio |
| Sample | [g] | [g] | [%] |
| CRECIA | 0.543 ± 0.022 | 0.086 ± 0.029 | 84 ± 5.8 |
| (One pair of | | | |
| two sheets) | | | |
| CA film P | 0.366 ± 0.035 | 0.067 ± 0.009 | 82 ± 1.8 |
| (Backside faces | | | |
| underside) | | | |
| Dialysis membrane | 0.367 ± 0.013 | 0.0983 ± 0.0025 | 73 ± 0.72 |
| (MWCO: 6000-8000) | | | |

The CA film P turned from opaque white into translucent white upon contact with Milli-Q water. Thus, it is considered that the increment in transparency and the enhancement of the passing light volume allows for an observation with a phase contrast microscope. Accordingly, it turned out that the CA film P is a porous cellulose acetate film having high transparency when it is wet.

### 1-4. Manufacturing of Dual-Layer Substrate Using Electrospinning.

There was fabricated a dual-layer substrate that enables temporal observation, where the substrate includes a cellulose acetate film layer that has a good water-absorbing property, becomes more transparent when it gets wet and was prepared in accordance with the section “1-3. Preparation of porous and highly transparent cellulose acetate film”; and a layer (gelatin fibers) for attaching cells thereon. The experimental methodology and the results are as described hereunder.

### 1-4-1. Experimental Methodology

A gelatin solution of powdered gelatine and hexafluoro-2-propanol (HFIP) was prepared in a sample tube to be a final concentration of 10 w/v %, which was stirred overnight under room temperature. On the next day, the gelatin solution was added into a syringe, which was installed on an electrospinning device (NANON). A porous cellulose acetate film was attached to its drum collector to which the fibers were spun. 3 mL of the gelatin solution was spun with the settings of: rotation speed of the drum collector being 2500 rpm, distance between the drum collector and the spinneret being 15 cm, moving speed of the spinneret being 10 mm/sec, moving width of 100 mm, applied voltage of 18 kV, spinning speed of 1.5 mL/h, and humidity of 30% or more. After spinning the fiber, the resultant dual-layer substrate was attached on an empty-and-deep bath having a rectangular shape, and air-dried for about two hours in a fume hood to remove the remaining solvent for performing drying of the gelatin fibers. Subsequently, a dish to which a suitable amount of 25% glutaraldehyde solution had been added was put into the bath to which the dual-layer substrate was attached, and the bath was sealed to perform vapor cross-linking overnight under room temperature. After performing the vapor cross-linking, it was air-dried for about two hours in a fume hood to remove the remaining glutaraldehyde. The gelatin was made insolubilized in this way by means of cross-linking by glutaraldehyde.

The insolubilization of the gelatin was confirmed under an environment that is analogous to the cell culture environment. First, the fabricated dual-layer substrate was formed into a suitable size (about 1 cm×1 cm) with scissors. Next, the dual-layer substrates were immersed into each solution of sterilized water and culture medium, and stored in an incubator (37° C., 5% CO₂) until 7 days later (until Day 7), 14 days later (until Day 14) and 21 days later (until Day 21) while replacing the solution every two days. Note that the expression “Day (numeral)” refers to a day that is counted in a way where the first day indicates “Day 0”. In order to observe the stored samples with a scanning electron microscope, they underwent dewatering processes with, in a sequential manner, 20%, 50%, 75% and 100% ethanol solutions for removing the moisture in, for example, the culture medium contained in the samples, and then underwent a freeze dehydration process with t-butyl alcohol (it was feezed by liquid nitrogen and dried for 6 hours under vacuum) for completely drying them.

Subsequently, an Au Sputtering (Target: Au, Discharge current: 35 mA, Sputtering time: 0.5 minutes) under a vacuum was performed for the observation with a scanning electron microscope. The morphology of the resultant gelatin fibers was observed from the obtained observed images and their fiber diameters were measured using ImageJ. Regarding the calculation of the fiber diameter, after selecting every 3 fields of view in a magnified image at 1000× for each sample, the diameters of 20 fibers at 3 locations per field of view were measured. That is, diameters at 60 locations per one field of view were measured. An average fiber diameter for all of the measured fibers and the standard deviation were calculated.

### 1-4-2. Results

FIG. 2 shows images observed by a scanning electron microscope of the dual-layer substrate on which fibers are spun. The fiber layer and the CA film P had good liquid permeabilities and therefore were found to be suitable for culture substrates.

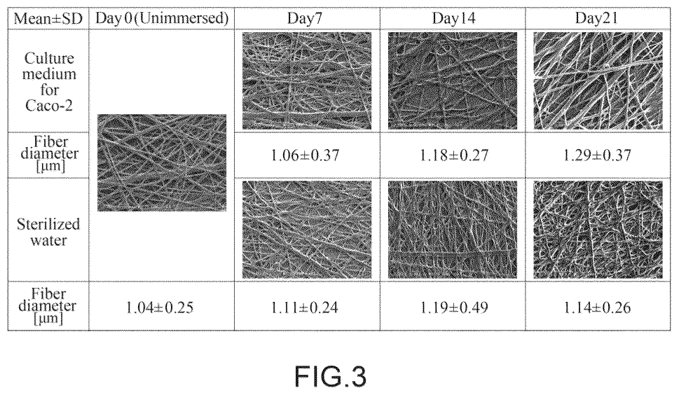

FIG. 3 shows observed images of the resultant dual-layer substrate which are taken by a scanning electron microscope. Since the diameters of the fibers remained virtually unchanged with an increment of the days having been immersed therein, it is considered that the fiber can be used for a culture that necessitates a long period of time.

### 1-4-3. Brief Summary

The Loeb-Sourirajan method was modified to establish fabrication conditions of porous cellulose acetate film. A cellulose acetate film that was porous and had a high transparency when it was wet had successfully been made, and gelatin fibers were spun on the film by electrospinning to fabricate a dual-layer substrate. The resultant dual-layer substrate was stable in terms of fiber diameter and its morphology under the cell culture condition (immersed in culture medium at 37° C. under 5% CO₂) and no fiber layer was exfoliated, which indicates that the dual-layer substrate can be used for a cell culture experiment.

### (Example 2) Fabrication of Intestinal Epithelial Tissue Model Using Dual-Layer of Cellulose Acetate Film and Gelatin

Caco-2 cells were subjected to either a submerged static culture (hereafter referred to as “submerged culture”) or air phase/liquid phase interface culture (hereafter referred to as “air-liquid interface culture”) using the dual-layer substrate that was prepared in accordance with the working example 1 and had good absorbency and a high transparency when it was wet to see if there can be made any temporal observation of a morphological change in Caco-2 cells until 21 days later (up to Day 21) after the culture. The experimental methodology and the results are as described hereunder.

### 2-1. Reagents

2-1-1. The reagents used for preparing the dual-layer substrate are as listed in the following.

(1) Nitta Gelatin, beMatrix* Gelatin LS-H (Alkaline-treated gelatin derived from porcine skin), Nitta Gelatin Inc., Osaka.

(2) 1,1,1,3,3,3-Hexafluoropropan-2-ol: Cat #PC4750, Apollo Scientific, UK.

(3) 25% glutaraldehyde solution: Cat #073-00536, FUJIFILM Wako Pure Chemical Corporation, OSAKA.

### 2-1-2. The Reagents Used for Cell Culture Examinations in the Dual-Layer Substrate are as Listed in the Following.

(1) Caco-2 cell of Human colon carcinoma (from ECACC): Cat #RCB0988, RIKEN BRC cell bank.

(2) Falcon® Petri Dish: Cat #351029, Corning Incorporated, the United States of America.

(3) Dulbecco's Modified Eagle Medium ((4.5 g/L Glucose) with L-Gln, without Sodium Pyruvate, liquid): Cat #08459-35, Nacalai Tesque Inc., Kyoto.

(4) Penicillin-Streptomycin: Cat #15140-122, Thermo Fisher Scientific Inc., Massachusetts, the United States of America.

(5) 0.5w/v % Trypsin-5.3 mmol/L EDTA-4Na Solution without Phenol Red (10x): Cat#208-17251, FUJIFILM Wako Pure Chemical Corporation, OSAKA.

(6) Dulbecco's phosphate buffered saline (10x): Cat #11482-15, Nacalai Tesque Inc., Kyoto

(7) Fetal Bovine Serum (Dominican Republic Origin): Cat #FB-1061/500, Lot #12868, Biosera

(8) MEM Non-Essential Amino Acids Solution(100x): Cat #06344-14, Nacalai Tesque Inc., Kyoto

### 2-1-3. Devises and Reagents Used for Cell Fixation and Fluorescent Staining are as Listed Below.

(1) Matsunami's glass bottom dish (dish diameter: q35 mm, glass diameter: q27 mm, glass thickness: No. 1S (0.16-0.19 mm)): Cat #D11040H, Matsunami Glass Ind., Ltd., Osaka.

(2) Alexa Fluor™ 568 Phalloidin: Cat #A12380, Thermo Fisher Scientific Inc., Massachusetts, the United States of America

(3) Hoechst 33342: Thermo Fisher Scientific Inc., Massachusetts, the United States of America

(4) 4% Paraformaldehyde Phosphate Buffer Solution: Cat #163-20145, FUJIFILM Wako

### Pure Chemical Corporation, OSAKA

(5) Triton® X-100: Cat #648466, Wako Pure Chemical Corporation, OSAKA

(6) Albumin, from Bovine Serum (BSA), Protease Free: Cat #018-15154, FUJIFILM Wako Pure Chemical Corporation, OSAKA

2-1-4. The Following Reagents were Used for the Dewatering/Freeze Dehydration Processing:

(1) Anhydrous ethanol: Cat #321-00025, FUJIFILM Wako Pure Chemical Corporation, OSAKA

(2) t-butyl alcohol: Cat #025-03396, FUJIFILM Wako Pure Chemical Corporation, OSAKA

### 2-2. Devices

2-2-1. The Following Devices were Used for the Preparation of the Dual-Layer Substrate.

(1) Electrospinning device: Cat #NANON-03, MECC CO., LTD., FUKUOKA.

(2) Tubeless Spinneret_75: Cat #S-TU/75, MECC CO., LTD., FUKUOKA.

(3) Drum Collector_q200W200 (Diameter 200 mm, Width 200 mm): Cat #C-DR/D200W200, MECC CO., LTD., FUKUOKA.

(4) Terumo Syringe® 1 mL for Tuberculin, Electron beam sterilized: Cat #SS-01T, Terumo Corporation, Tokyo.

(5) Terumo Injection Needle (electron beam sterilized, 22G×1 1/4), Terumo Corporation, Tokyo.

(6) Crecia EF Hand Towel Soft 100 Piece: NIPPON PAPER CRECIA CO., LTD., Tokyo

(7) 3M™ Masking Tape 243J Plus: 3M Japan Limited, Tokyo.

(8) Electric Drying Oven: Cat #DRA330DA, ADVANTECH CO. LTD., Tokyo

(9) Magnetron sputtering system: Cat #MSP-10, Vacuum device Corp., Ibaraki.

(10) Scanning electron microscope: Cat #VE-9800, KEYENCE CORPORATION, Osaka.

2-2-2. The following devices were used for cell culture experiments on the dual-layer substrate.

(1) Cabinet For Biohazard Measure Class II Type A: Cat #MHE-131AJ, SANYO Electric Co., Ltd., Osaka

(2) Table top clean bench (KVM type): Cat #KUM-756, AIRTECH JAPAN, Ltd., Tokyo

(3) Digital microscope: Cat #MS-200, Olympus Corporation, Tokyo

(4) Phase contrast microscope: Cat #Eclipse TS100, Nikon Corporation, Tokyo

(5) Inverted Research Microscope: Cat #IX81, Olympus Corporation, Tokyo

(6) Confocal Laser Scanning Microscope: Cat #FLUOVIEW FV1000, Olympus Corporation, Tokyo

### 2-3. Temporal Observation in Submerged Static Culture

A submerged static culture was performed to see if Caco-2 cells can be attached to and/or grown on the dual-layer substrate consisting of a porous cellulose acetate film layer and gelatin fibers. The details are as shown below.

### 2-3-1. Experimental Methodology

A dual-layer substrate was prepared in accordance with the method as explained in the working example 1. The prepared dual-layer substrate was cut with scissors into a size of 2.5 cm×2.5 cm. The substrate was immersed in a culture medium to remove the unreacted glutaraldehyde on this substrate, which was then stored in an incubator (37° C., 5% CO₂) (this process will be referred to as “pre-incubation” in the following). The amount of the culture medium was set as 1.6 mL per 1 cm² of dual-layer substrate. Then, 500 μL of cell suspension in which the suspension had a seeding concentration of 5.53×10⁴ cells/cm² of Caco-2 (Passage number 58) in the logarithmic growth phase was prepared. The cell culture site was set as within the inner side of a silicone square ring having a size of 2 cm×2 cm, i.e., 4 cm², to cover the periphery of the dual-layer substrate with the silicone square ring to prevent the Caco-2 cells and the culture medium from being leaked therefrom. A 500 μL of the cell suspension was seeded in the 4 cm² site, where after this cell culture site was stored in an incubator (37° C., 5% CO₂) overnight until the Caco-2 were attached on the dual-layer substrate. On the next day, 200 μL of culture medium was added to the inside of the silicone square ring, which was stored overnight in the incubator (37° C., 5% CO₂). On the next day, the silicone square ring is removed therefrom and 20 μL of culture medium is added to immerse the dual-layer substrate in the culture medium. After that, culture media replacement and observation by a phase contrast microscope were carried out for every 2 to 3 days while culturing the same for 21 days (up to Day 21).

The samples having been cultured for 21 days underwent cell fixation by 4% paraformaldehyde (room temperature, for 15 minutes.) and stored in a refrigerator at −4° C. until it was subjected to the staining. As to the staining, 0.15% of Triton X-100(1×PBS dilution) was used to perform cell membrane permeabilization and 5% of BSA (1×PBS dilution) was used to perform the blocking (room temperature, for 30 minutes). Then, Alexa Fluor 568 Phalloidin (1:400, 1% BSA dilution) was reacted for 30 minutes under room temperature and Hoechst 33342(1:1000, 1×PBS dilution) was reacted for 15 minutes under room temperature to stain F-actin and nucleus. Since the stained samples were dual-layer substrates having highly transparent CA films P when they are wet, they may be observed as they are. Meanwhile, the observation was performed with the substrate being flipped for the purposes of comparison because the prior dual-layer substrate with paper enables observation by flipping the substrate so that the cell attachment side is in contact with the bottom surface of the dish and using a confocal microscope for fluorescent observation. The conditions for the observation (such as Gain, offset) were not constant among the samples, and therefore the observation was carried out by tuning the conditions such that their morphologies can be clearly observed.

### 2-3-2. Results

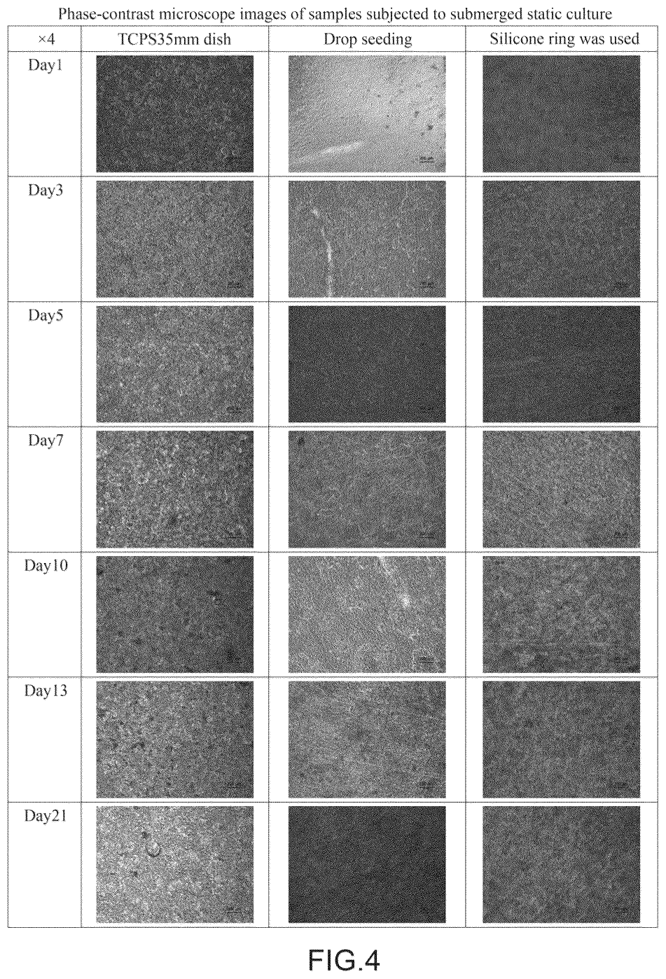

A dual-layer substrate having been made by spinning 3 mL of gelatin fiber on the CA film P as shown in example 1 was used to seed Caco-2 cells, which were subjected to submerged static culture for 21 days (up to Day 21) and observed using a phase contrast microscope, and the results of observation at Day 1, Day 3, Day 5, Day 7, Day 10, Day 13 and Day 21 are as shown in FIG. 4.

The cells were confirmed to be attached thereto and their growth situations were allowed to be temporally observed when compared to the cells having been seeded as controls on a culture dish (TCPS (Tissue-culture-treated polystyrene) 35 mm dish). Nevertheless, the cells generally formed a planer structure and no three-dimensional structures peculiar to the intestinal epithelia were observed. Regarding the cell seeding method, drop seeding caused the cells and culture medium to flow out of the fibers, and the number of the adhered cells was lower than the one when a silicone ring was used. For this reason, it was determined that seeding methods employing a silicone square ring were carried out in the subsequent examinations.

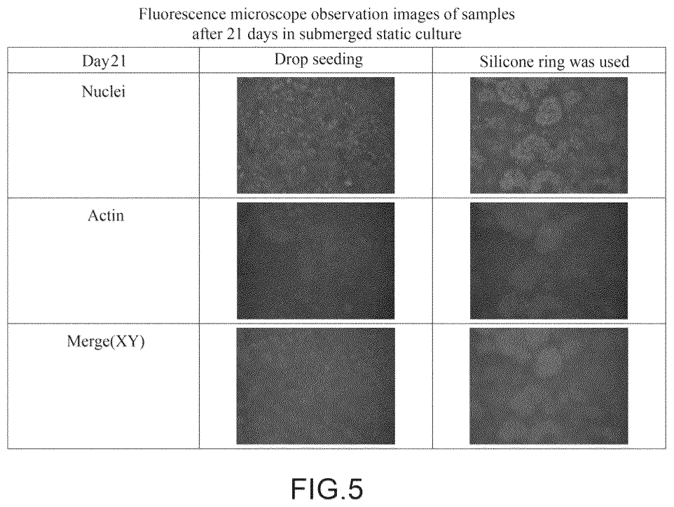

Temporal observation with a phase contrast microscope was made possible, and the actin and nuclei were stained to confirm if the cells actually existed by making an observation with a fluorescence microscope whose results are as shown in FIG. 5. From this result, actin and nuclei were confirmed, which indicates that the cell can be cultured in the dual-layer substrate consisting of the CA film P and gelatin fibers.

### 2-4. Temporal Observation in Air-Liquid Interface Culture.

Next, a temporal observation in air-liquid interface culture was performed to see if three dimensional structures peculiar to the intestinal villus was produced and to see if it could be temporally observed using a phase contrast microscope.

### 2-4-1. Experimental Methodology

A dual-layer substrate was prepared using methods of those as explained in the section of 2-3. The amounts of gelatin fibers were set as 1.5 mL, 2.0 mL, 2.5 mL and 3.0 mL to examine the relationship between the light transmittance of dual-layer substrate and the amount of gelatin fibers. Moreover, a dual-layer substrate of the prior art (non-patent document 3) consisting of paper and 3.0 mL of gelatin fiber was also prepared.

The prepared dual-layer substrate was cut with scissors into a size of 2.5 cm×2.5 cm. The substrate was subjected to the pre-incubation for one night or more. The amount of the culture medium was set as 1.6 mL per 1 cm² of dual-layer substrate. Then, 500 μL of cell suspension in which the suspension had a seeding concentration of 5.53×10⁴ cells/cm² of the Caco-2 (passage number 58) in the logarithmic growth phase was prepared. The cell culture site was set as within the inner side of a silicone square ring having a size of 2 cm×2 cm, i.e., 4 cm², to cover the periphery of the dual-layer substrate with the silicone square ring to prevent the Caco-2 cells and the culture medium from being leaked therefrom. A 500 μL of the cell suspension was seeded in that 4 cm² site, where after this cell culture site was stored in an incubator (37° C., 5% CO₂) overnight until the Caco-2s were attached on the dual-layer substrate. On the next day, 200 μL of culture medium was added to the inside of the silicone square ring, which was stored overnight in the incubator (37° C., 5% CO₂). On the next day, the silicone square ring is removed therefrom and 10 μL of culture medium is added thereto to immerse the dual-layer substrate in the culture medium to incubate the same overnight. On the next day, a silicone U-shape strand in which a 0.5 cm width slit was made in only one of the sides was placed on a φ 100 mm dish, and 25 mL culture medium was put thereinto. A dual-layer substrate of cellulose acetate film having been seeded with the cells was placed on that silicone U-shape strand to stablely hold it with a silicone square ring from above. After that, culture media replacement and observation by a phase microscope were carried out every 2 to 3 days while culturing the same for 21 days (up to Day 21).

The samples having been cultured for 21 days underwent cell fixation by 4% paraformaldehyde (room temperature, 15 minutes) and stored in a refrigerator at −4° C. until it was subjected to staining. As to the staining, 0.15% Triton X-100 (1×PBS dilution) was used to perform cell membrane permeabilization, and 5% BSA (1×PBS dilution) was used to perform blocking (room temperature, for 30 minutes). Then, Alexa Fluor 568 Phalloidin (1:400, 1% BSA dilution) was reacted for 30 minutes at room temperature and Hoechst 33342 (1:1000, 1×PBS dilution) was reacted for 15 minutes at room temperature to stain F-actin and nuclei. Since the stained samples were dual-layer substrates having highly transparent CA films P when they are wet, they may be observed as they are. Meanwhile, the observation was performed with the substrate being flipped for comparison because the comparative example of a dual-layer substrate with paper enables observation of a confocal microscope for fluorescent observation by flipping the substrate so that the cell attachment side is in contact with the bottom surface of the dish. The conditions for the observation (such as Gain and offset) were not constant among the samples, and therefore the observation was carried out by tuning the conditions such that their morphologies could be clearly observed.

### 2-5. Results

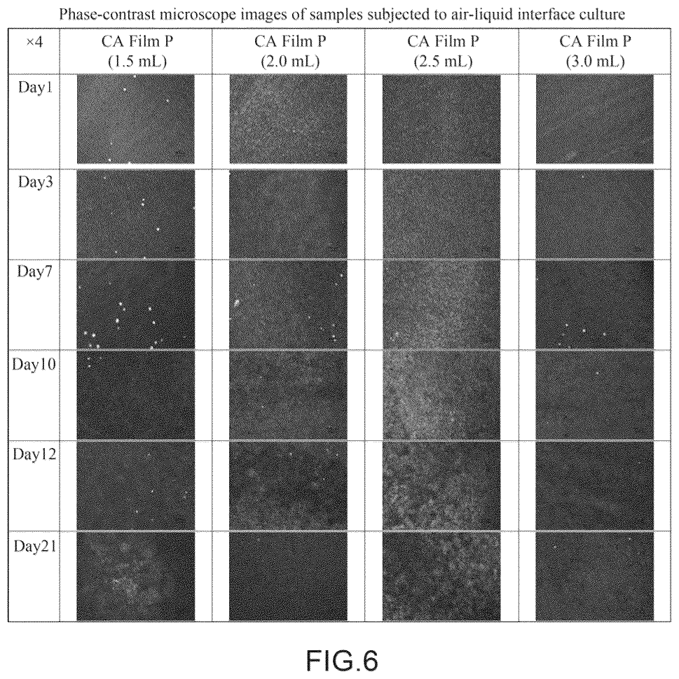

A dual-layer substrate of the CA film P was used to perform an air-liquid interface culture for 21 days (up to Day 21), which was observed using a phase contrast microscope, and the results of the observation are as shown in FIG. 6.

The cells from about Day 10 onward showed cells that were not in a monolayer but in an convex shape.

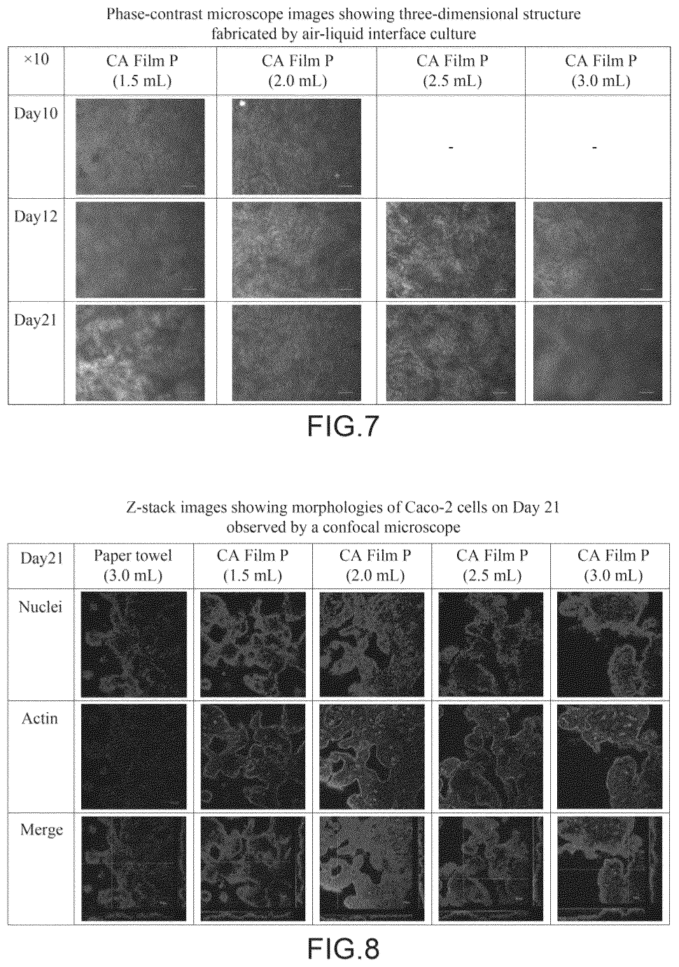

FIG. 7 illustrates phase contrast images with an objective lens of magnitude 10 which clarifies the convex structure of the cells. It displayed structures analogous to intestinal villus, viewed from above, having three-dimensional structures that are present in the intestinal epithelia. It showed the highest light transmittance and many three-dimensional structures when 2.5 mL of gelatin fiber was used.

The samples having been cultured for 21 days (up to Day 21) underwent cell fixation and staining to see if the objects observed in the phase contrast image had three-dimensional structures analogous to the intestinal villus, which were then subjected to Z-stack imaging using a confocal laser microscope. The results are as shown in FIG. 8. Meanwhile, for the sake of comparison purposes, an air-liquid interface culture was also made on a dual-layer substrate of paper (paper towel) on which 3 mL of gelatin fibers was spun, and the observation was carried out. The Z-stack image illustrates that the dual-layer substrate having been made of a CA film P was formed of a monolayer analogous to the intestinal villus as is the case with the paper dual-layer substrate, and a three-dimensional structure of a hemisphere inside of which being hollow was confirmed.

It should therefore be inferred that the structures observed in the phase contrast image in FIG. 7 were three-dimensional structures of the intestinal villus, and therefore temporal observation by a phase contrast microscope was enabled.

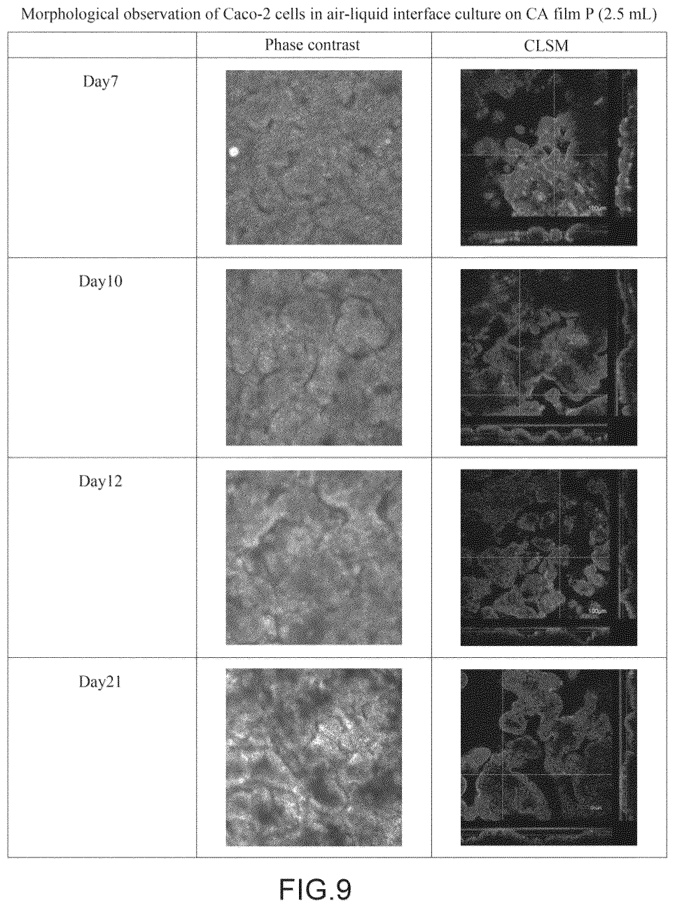

Caco-2 cells were cultured on a dual-layer substrate in which 2.5 mL of gelatin fibers were spun on a CA film P. FIG. 9 illustrates images showing the morphologies peculiar to the intestinal epithelial cells which were observed by a phase contrast microscope and a confocal laser scanning microscope (CLSM). Since gelatin fiber exhibits red autofluorescence, it had been found from Day 7 onward that actin and nuclei were present on the gelatin fibers. From around Day 10 onward, a void started to be formed between the gelatin fibers and nucleus, and around Day 12, a monolayer of the nucleus was elevated to form three-dimensional structures of hemispheres inside of which being hollow, which is peculiar to the intestinal epithelial tissue.

This unique three-dimensional structure on Day 12 had progressively matured until Day 21.

### 2-5. Brief Summary

As explained above, a dual-layer substrate of the CA film P having been prepared in the working example which has high transparency and good water absorbency was used to study if Caco-2 could realistically be cultured in a submerged static culture or to study if a morphological change of the Caco-2 cells could be temporally observed over a period of 21 days (up to Day 21) of culture by performing an air phase/liquid phase interface culture. As a result, the Caco-2 cells were able to be cultured in a submerged static culture on a dual-layer substrate of the CA film P. As to the morphology of the cells, although only a planer two-dimensional structure had been formed, a temporal observation was made possible. Moreover, a three-dimensional hemispherical structure analogous to the case of paper dual-layer substrate was formed when an air/liquid interface culture was employed, and their three-dimensional structures were able to be temporally observed using a phase contrast microscope. In view of these results, the issue of two-dimensional structure, which had been an issue associated with the prior art intestinal epithelia model, was able to be resolved; in addition, the issue of the inability to conduct temporal observation by an optical microscope, which had been an issue in fabricating an intestinal epithelia model by a paper dual-layer substrate, was also able to be resolved. That is, there had successfully been achieved fabrication of an in vitro intestinal epithelial tissue model that can be temporally observed by an optical microscope in a continuous manner.

### (Example 3) Structure Observation of Intestinal Epithelia Model Using Scanning Electron Microscope (SEM)

A scanning electron microscope (SEM) was used to observe a structure of the intestinal epithelia model as prepared in example 2, which was compared with an intestinal epithelial tissue obtained by a culture using a culture insert that had been conventionally used.

### 3-1. Experimental Methodology

Caco-2 cells were seeded on a culture medium to be 70% confluence. The cells were cultured with the total of four conditions of: using an air-liquid interface culture or a submerged culture on a dual-layer substrate of the CA film P and gelatin fibers; and using an air-liquid interface culture or a submerged culture on a cell culture insert (Corning, Cat #3460, USA). They were each transferred into an air-liquid interface culture or a submerged culture three days later after the culture and the culture media were replaced every 1 to 2 days. Each sample was fixed by 4% paraformaldehyde Phosphate Buffer Solution (Cat #163-20145, FUJIFILM Wako Pure Chemical Corporation, OSAKA) at five time points of 5 days later after the culture (Day 5) (two days later after having been changed into the respective culture conditions), 7 days later after the culture (Day 7), 10 days later after the culture (Day 10), 12 days later after the culture (Day 12) and 21 days later after the culture (Day 21), which were subjected to alcohol replacement process under a conventional method to dewater them, and then air dried (for one or more days), after which they were subjected to Au Sputtering to observe a morphological change in the respective samples by using a scanning electron microscope.

### 3-2. Experimental Results

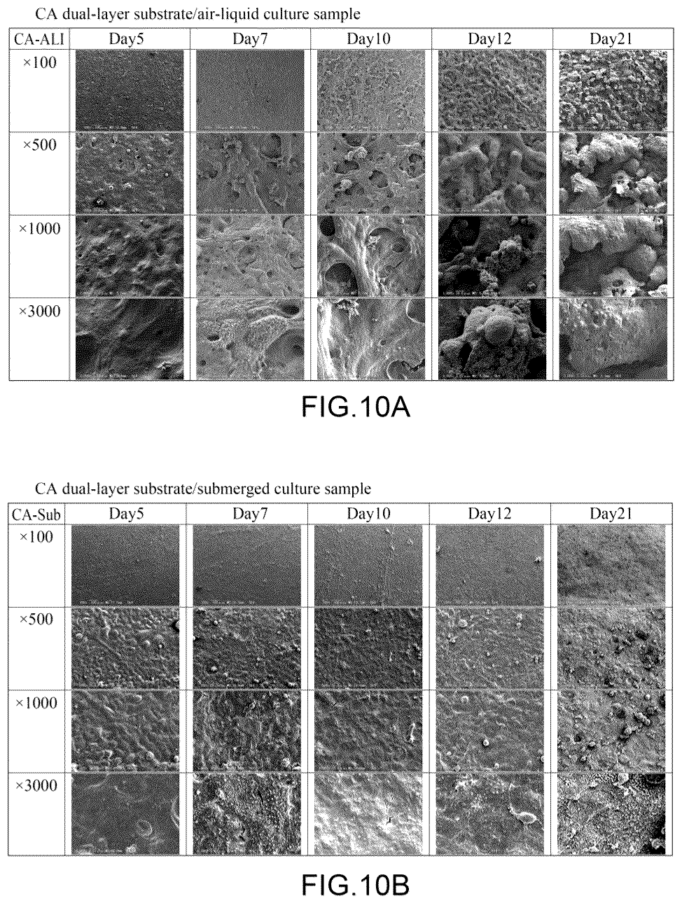

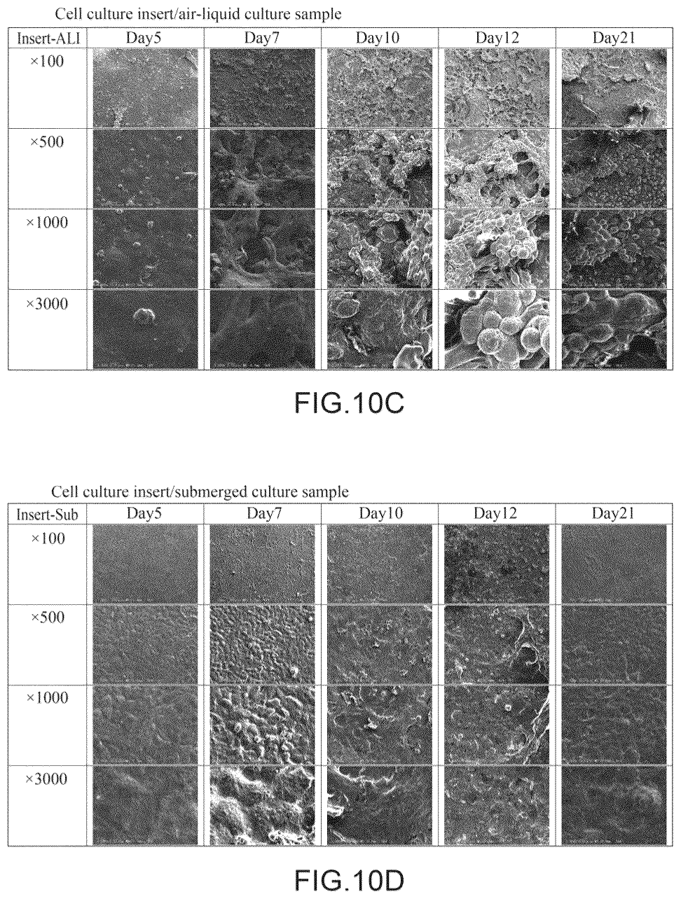

FIG. 10A illustrates SEM images of the samples each obtained with an air-liquid interface culture on a dual-layer substrate of CA film Player and gelatin fibers. As the culture progressed, three-dimensional structures resembling intestinal villus were observed to be formed. Microvilli-like structures were also observed at high magnification.

FIG. 10B illustrates SEM images of the samples each obtained with a submerged culture on a dual-layer substrates of CA film P layer and gelatin fibers. No three-dimensional structures resembling intestinal villus were observed to form at any culture days. However, microvilli-like structures were observed at high magnification.

FIG. 10C illustrates SEM images of the samples each obtained by an air-liquid interface culture using a cell culture insert. The samples were flat at Day 5. Nevertheless, from Day 7 onward, elevations were observed to form. These structures were closer to planarly structures which resemble scabs and were hard to recognize as intestinal villi having the shapes of fingers. It may also be inferred that these structures were potentially of a mucus layer rather than of the structure.

FIG. 10D illustrates SEM images of the samples each obtained by a submerged culture using a cell culture insert. No three-dimensional structures resembling intestinal villus were observed to form at any culture days. However, microvilli-like structures were observed at high magnification

### (Example 4) Alanine Aminopeptidase (ANPEP) Activity Observation in Intestinal Epithelial Tissue Model

Digestive enzymes are produced in biological intestinal epithelial tissue. Accordingly, activity progress of alanine aminopeptidase (ANPEP)—a digestive enzyme—was temporally observed to see the enzyme activity which is a functionality of the intestinal epithelial tissue model as prepared in example 2.

### 4-1. Experimental Methodology

Caco-2 cells were seeded on a culture medium to be 70% confluence. Cells were cultured with the total of four conditions of: using an air-liquid interface culture or a submerged culture on a dual-layer substrate of the CA film P and gelatin fibers; and using an air-liquid interface culture or a submerged culture on a cell culture insert. They were each transferred into an air-liquid interface culture or a submerged culture three days later after the culture and the culture media were replaced every 1 to 2 days (N=1 respectively). Alanine aminopeptidase (ANPEP) activities were measured at five time points of 5 days later after the culture (Day 5) (two days later after having been changed into the respective culture conditions), 7 days later after the culture (Day 7), 10 days later after the culture (Day 10), 12 days later after the culture (Day 12) and 21 days later after the culture (Day 21). The measurement of ANPEP activities was in accordance with the method of “Shim K.-Y., et al” which utilizes light absorbance determination of p-nitroaniline produced by the hydrolysis of L-Alanine-4-nitroanilide (Shim K.-Y., et al, Biomed Microdevices 2017; 19:37).

### 4-2. Experimental Results

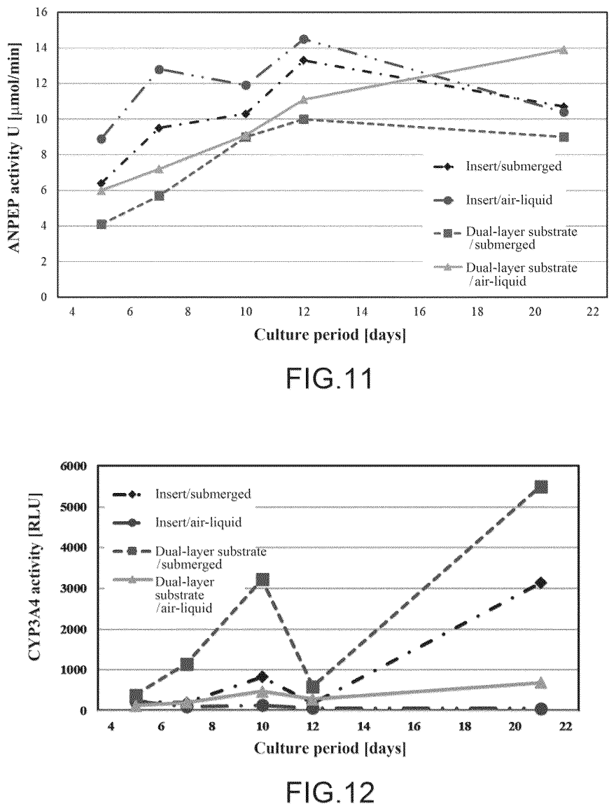

FIG. 11 illustrates the experimental results. The ANPEP activities continued to increase until 12 days later after the culture (Day 12) under all 4 conditions of: using an air-liquid interface culture or a submerged culture on a dual-layer substrate of the CA film P and gelatin fibers; and using an air-liquid interface culture or a submerged culture on a cell culture insert. The activity on the dual-layer substrate of the CA film P and gelatin fibers using the air-liquid interface culture was further increased on the day after 21 days of culture (Day 21) while the activities were comparable to those on the day after 12 days of culture (Day 12) when a submerged culture on a dual-layer substrate of the CA film P and gelatin fibers, an air-liquid interface culture in a cell culture insert and a submerged culture in a cell culture insert were employed. When comparing among culture methods, it was observed that an air-liquid interface culture results in an enhanced activity compared to the one of the submerged culture.

It was demonstrated that the intestinal epithelial tissue model having been fabricated using a dual-layer substrate of the CA film P and gelatin fibers exhibits ANPEP activity which is analogous to the activity of a biological intestinal epithelial tissue.

### (Example 5) CYP3A4 Activity Observation in Intestinal Epithelial Tissue Model

A biological intestinal epithelial tissue exhibits CYP3A4 activity. Accordingly, activity progress of CYP3A4—a digestive enzyme—was temporally observed to see the enzyme activity which is a functionality of the intestinal epithelial tissue model as prepared in example 2.

### 5-1. Experimental Methodology

Caco-2 cells were seeded on a culture medium to be 70% confluence. Cells were cultured with the total of four conditions of: using an air-liquid interface culture or a submerged culture on a dual-layer substrate of the CA film P and gelatin fibers; and using an air-liquid interface culture or a submerged culture on a cell culture insert. They were each transferred into an air-liquid interface culture or a submerged culture three days later after the culture and the culture media were replaced every 1 to 2 days (N=1 respectively). CYP3A4 activities were measured at five time points of: 5 days later after the culture (Day 5) (two days later after having been changed into the respective culture conditions), 7 days later after the culture (Day 7), 10 days later after the culture (Day 10), 12 days later after the culture (Day 12) and 21 days later after the culture (Day 21). The measurement of CYP3A4 activities was in accordance with the method of “Shim K.-Y., et al” which utilizes Luciferin-IPA as a substrate to measure the luminescence of the produced D-Luciferin when treating with a luciferin fluorescence detecting solution that contains luciferase (See, Shim K.-Y., et al, Biomed Microdevices 2017; 19:37).

### 5.2. Experimental Results

FIG. 12 illustrates the experimental results. On 21 days later after the culture (Day 21), the intensities of CYP3A4 activities were in the order from highest to lowest as: a submerged culture on a dual-layer substrate of the CA film P and gelatin fibers, a submerged culture in a cell culture insert, an air-liquid interface culture on a dual-layer substrate of the CA film P and gelatin fibers, and an air-liquid interface culture in a cell culture insert, and no CYP3A4 activity enhancement was observed in an air-liquid interface culture in a cell culture insert. That is, it was observed that the submerged culture resulted in higher activities than those of the air-liquid interface culture when compared among culture methods.

It was demonstrated that the intestinal epithelial tissue model having been fabricated using a dual-layer substrate of the CA film P and gelatin fibers exhibits CYP3A4 activity which is analogous to the activity of a biological intestinal epithelial tissue.

### (Example 6) Mucus Production Observation on Intestinal Epithelial Tissue Model

A biological intestinal epithelial tissue produces mucus. Accordingly, a study of mucus production capability was conducted on the intestinal epithelial tissue model having been fabricated using a dual-layer substrate of the CA film P and gelatin fibers.

### 6.1 Experimental Methodology

Caco-2 cells were seeded on a culture medium to be 70% confluence. Cells were cultured with the total of four conditions of: using an air-liquid interface culture or a submerged culture on a dual-layer substrate of the CA film P and gelatin fibers; and using an air-liquid interface culture or a submerged culture on a cell culture insert. They were each transferred into an air-liquid interface culture or a submerged culture three days later after the culture and the culture media were replaced every 1 to 2 days (N=3 respectively).

Each culture sample was fixed by 4% paraformaldehyde Phosphate Buffer Solution (Cat #163-20145, FUJIFILM Wako Pure Chemical Corporation, OSAKA) at five time points of 5 days later after the culture (Day 5) (two days later after having been changed into the respective culture conditions), 7 days later after the culture (Day 7), 10 days later after the culture (Day 10), 12 days later after the culture (Day 12) and 21 days later after the culture (Day 21), and then subjected to the alcian blue staining to perform an image analysis by an observation using a phase contrast microscope to thereby estimate the mucus production capability. The alcian blue staining was carried out by letting them stand at room temperature for 12 hours in a 3% acetic acid solution of 0.1(w/v) % alcian blue, and then washing them twice with the PBS.

As to the observation by the phase contrast microscope, the image analysis was performed using the following method by obtaining images of 10 fields of view for each sample at 20-fold magnification with the light intensity and white balance being fixed at a constant value. The dual-layer substrate of only the cells and fiber layer was observed with the CA film peeled off.

The image analysis was carried out using the following processes.

(i) An original image was transformed into the HSV (H: Hue; S: Saturation=brilliance of a color; V: Value=brightness of a color) using Image J.

(ii) An image of “H” was used to exclusively specify and extract the blue area (Threshold: 0.5 to 0.6) which is an area (=mucus) having been stained by alcian blue (image H′).

(iii) The image H′ having been treated with the process (ii) is superimposed on the image of V, and a blue area is exclusively extracted with respect to the brightness, which was then reversed such that a darker brightness turned into to have a higher brightness value.

(vi) The number of pixels per brightness value was calculated from the histogram of the V+H′ image having been treated with the processes (i) and (ii). (The brightness was expressed at 256 steps ranging from 0 to 256 since the image was of 8-bit. Here, the higher the brightness value was, the more the blue color intensified)

(v) 30 pictures were subjected to image processing for one culture condition because 10 fields of view were taken per one sample where three samples were used for one culture condition.

(vi) After having calculated the respective brightness values and the number of pixels corresponding to the respective brightness values, the multiplied values of them at each value were summed to determine the mucus production quantity in a given condition as:

$$Mucus production quantity = \qquad [ Formula 1 ]$$

$${\sum }_{i = 0}^{254} Brightness value ( i ) \times the number of pixels for the brightness value$$

Further, for ease of understanding, the mucus production quantities were also expressed in terms of percentage as:

$$Mucus production quantity ( \% ) = \frac{Mucus production quantity of each sample}{\begin{matrix}The highest quantity among the mucus production \\ quantities in the present experiments\end{matrix}} \times 100 \qquad [ Formula 2 ]$$

### 6-2. Experimental Results

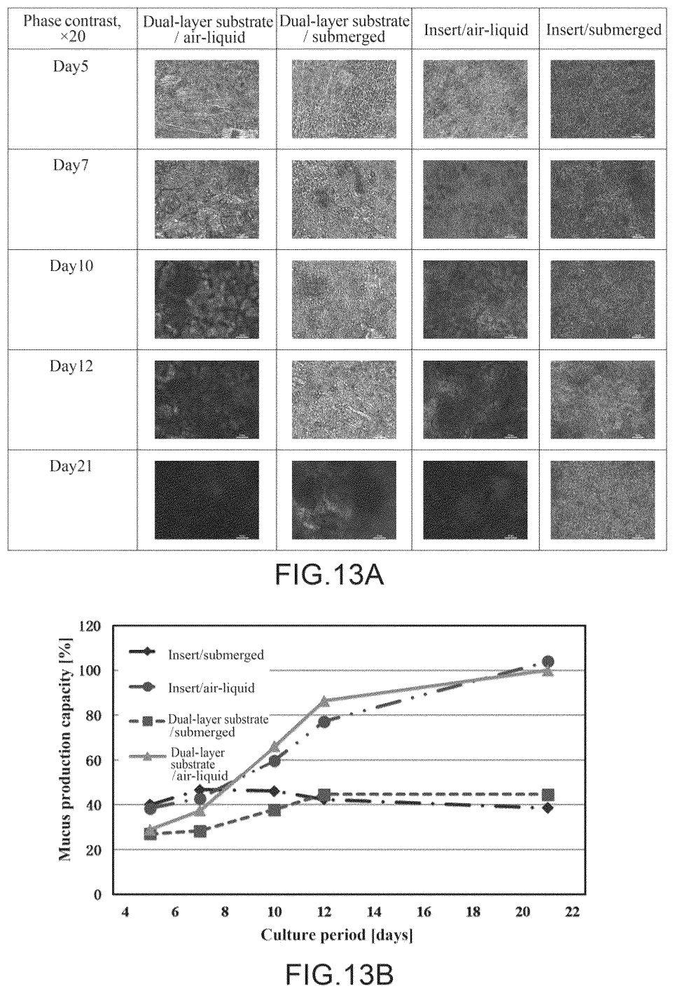

FIG. 13A shows representative examples of phase contrast microscope images of the respective samples at the respective time points. FIG. 13B shows temporal progress of the mucus production capacity for each experimental group, which was obtained by calculating it in terms of mucus production quantity.

As shown by the microscope images in FIG. 13A, when visually compared among the culturing methods, since the color of blue became more intensified for those of the air-liquid interface culture than those of the submerged culture as the culturing progressed day by day, it was found that an air-liquid interface culture led to a higher mucus production quantity than the one of a submerged culture. Further, as shown by the figure (FIG. 13B) showing the temporal progresses of mucus production capacity, which were obtained by analyzing and quantifying the microscope images, it was shown that the mucus production quantity became more increased for the samples of air-liquid interface culture than the samples of submerged culture as the culturing got progressed day by day. No difference in mucus production quantity was found among the culture substrates.

As shown from the results in the above, a mucus production capacity analogous to the one of biological intestinal epithelial tissue had been found in the intestinal epithelial tissue model having been fabricated using a dual-layer substrate of the CA film P and gelatin fibers.

### (Example 7) Evaluation of Barrier Function of Intestinal Epithelial Tissue Model

In a biological intestinal epithelial tissue, there is found a barrier function by which the passage of substances such as ions between apical and basolateral sides of a cell is regulated by tight junctions. A transepithelial electrical resistance (TEER) is measured as a method for evaluating barrier function in an epithelial tissue. Accordingly, the TEER value was measured on the intestinal epithelial tissue model having been prepared using the transparent dual-layer substrate according to the present invention to evaluate the barrier function.

### 7.1 Experimental Methodology

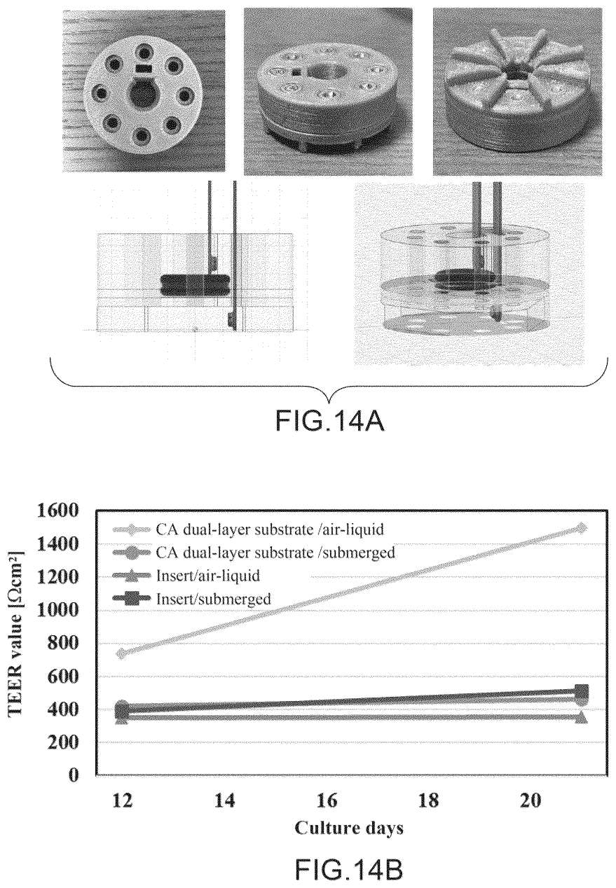

Caco-2 cells were seeded on a culture medium to be 70% confluence. Cells were cultured with the total of four conditions of: using an air-liquid interface culture or a submerged culture on a dual-layer substrate of the CA film P and gelatin fibers; and using an air-liquid interface culture or a submerged culture on a cell culture insert. They were each transferred into an air-liquid interface culture or a submerged culture three days later after the culture and the culture media were replaced every 1 to 2 days (N=3 respectively). TEER values were measured at two time points on the day of 12 days later after the culture (Day 12) (10 days later after having been changed into the respective culture conditions) and the day of 21 days later after the culture (Day 21) (19 days later after having been changed into the respective culture conditions). The transparent dual-layer substrate according to the present invention cannot be mounted on a commercially available TEER device as it is. For this reason, attached thereto was a measurement chamber (which is divided into upper and lower parts to sandwich the substrate there between) as shown in FIG. 14A and having been originally fabricated using a 3D printer. Since this chamber provides spaces for attaching electrodes of a commercially available TEER device, the measurement was conducted while a testing electrode (MERSSTX04) and an adjustable electrode (MERSSTX03) being attached on the Millicell ERS-2 Epithelial Volt-0 hm Meter manufactured by Millipore. During the measurement, the upper and lower chamber spaces were filled with a culture medium to perform the measurement.

The TEER values were measured using the following formula:

$$TEER value = ( {R}_{all} - {R}_{blank} ) \times S ,$$

where Ran is a measured value of resistance of a sample in which a dual-layer substrate was used to culture cells, Rblank is a measured value of resistance thereof in which a dual-layer substrate is solely used without culturing cells, and S is an effective culture area with 1.12 cm² for the cell culture insert and 1 cm² for the dual-layer substrate.

### 7-2. Experimental Results

FIG. 14B shows average values of the experimental values where N=3. The intestinal epithelial tissue model having been fabricated on the transparent dual-layer substrate by air-liquid interface culture exhibited a significantly high TEER value at the time points on the day of 12 days later after the culture (Day 12) and the day of 21 days later after the culture (Day 21) compared to the other tissue models fabricated by the other culture methods. Meanwhile, it has been reported that the reported TEER values of an intestinal epithelial tissue model in which Caco-2 cells were cultured by a cell culture insert for use in a substance permeability assay were about 100 to 850 Ohm*cm² (L.-F. Blume, et al., Pharmazie 65 (2010) 1, 19-24). Compared to these values, the intestinal epithelial tissue model having been fabricated on the dual-layer substrate by air-liquid interface culture exhibited a significantly high TEER value.

It was therefore demonstrated that the intestinal epithelial tissue model having been fabricated using the dual-layer substrate of the CA film P and gelatin fibers exhibits a high barrier function which is analogous to the function of a biological intestinal epithelial tissue.

We claim:

1-12. (canceled)

13. A transparent dual-layer substrate for culturing a cell and/or a tissue by air-liquid interface culture, comprising a porous cellulose derivative membrane on which polymer microfibers are spun and laminated, wherein the porous cellulose derivative membrane is light permeable under a wet condition, wherein the transparent dual-layer substrate is manufactured by a method comprising the steps of:
1) dissolving a cellulose derivative in an organic solvent to prepare an organic solvent solution of the cellulose derivative;
2) coating a substrate with the organic solvent solution of the cellulose derivative and drying it to prepare a cellulose derivative membrane;
3) drying the cellulose derivative membrane for a prescribed period of time, immersing the membrane in hot water for a prescribed period of time, immersing the membrane in cold water for another prescribed period of time, and then peeling the membrane from the substrate to prepare a porous cellulose derivative membrane; and
4) spinning polymer microfibers on the porous cellulose derivative membrane by electrospinning to laminate the porous cellulose derivative membrane with the polymer microfibers,
wherein coating thickness in the step 2) is from 150 to 200 μm, and an upper half of the membrane has an average porous diameter of 0.306 μm while a lower half of the membrane has an average porous diameter of 0.515 μm.

14. The transparent dual-layer substrate according to claim 13, wherein the cell is an intestinal epithelial cell or an epithelial cell of another tissue or organ or the cell is of epidermis cell, and wherein the tissue is an intestinal epithelial tissue or another epithelial tissue or the tissue is an epidermis tissue.

15. A biological tissue model involving air-liquid interface culture of a cell and/or a tissue on polymer microfibers laminated on a porous cellulose derivative membrane with the aid of the transparent dual-layer substrate according to claim 13.

16. The biological tissue model according to claim 15, wherein the cell is an intestinal epithelial cell or an epithelial cell of another tissue or organ or the cell is of epidermis cell, wherein the tissue is an intestinal epithelial tissue or another epithelial tissue or the tissue is an epidermis tissue, and wherein the biological tissue model is selected from an intestinal epithelial tissue model, an epithelial tissue model of another epithelial tissue and an epidermis tissue model.

17. The biological tissue model according to claim 16, wherein the intestinal epithelial tissue model expresses intestinal villus structure, microvillus structure, digestive enzyme activity, drug-metabolizing enzyme activity, mucus production capacity, barrier function and/or alkaline phosphatase (ALP).

18. A method of fabricating a biological tissue model, wherein the method comprises culturing a cell and/or a tissue by air-liquid interface culture on polymer microfibers using the transparent dual-layer substrate according to claim 13.

19. The method of fabricating a biological tissue model according to claim 18, wherein the cell is an intestinal epithelial cell or an epithelial cell of another tissue or organ or the cell is of epidermis cell, wherein the tissue is an intestinal epithelial tissue or another epithelial tissue or the tissue is an epidermis tissue, and wherein the biological tissue model is selected from an intestinal epithelial tissue model, another epithelial tissue model and an epidermis tissue model.

20. The method of fabricating a biological tissue model according to claim 19, wherein the intestinal epithelial tissue model expresses intestinal villus structure, microvillus structure, digestive enzyme activity, drug-metabolizing enzyme activity, mucus production capacity, barrier function and/or alkaline phosphatase (ALP).
